(19) United States
(12) Patent Application Publication
(10) Pub. No.: US 2015/0353447 A1
(43) Pub. Date: Dec. 10, 2015

(54) Transalkylation of Heavy Aromatic Hydrocarbon Feedstocks

(71) Applicant: ExxonMobil Chemical Patents Inc., Baytown, TX (US)

(72) Inventors: Jeevan S. Abichandani, Houston, TX (US); Gary D. Mohr, Sunset, SC (US); Robert G. Tinger, Friendswood, TX (US)

(21) Appl. No.: 14/717,023

(22) Filed: May 20, 2015

Related U.S. Application Data
(60) Provisional application No. 62/007,556, filed on Jun. 4, 2014.

(51) Int. Cl. C07C 4/18; B01J 29/70; B01J 29/44; B01J 29/40; C07C 7/148; C07C 6/06

(52) U.S. Cl. CPC C07C 4/18; C07C 7/1485; C07C 6/06; B01J 29/44; B01J 29/40; B01J 29/7034; B01J 29/7042; C07C 2529/44; C07C 2529/40; C07C 2529/70

(57) ABSTRACT

A process for producing xylene comprises contacting a first feed comprising C₉₊ aromatic hydrocarbons, at least one C₆-C₇ aromatic hydrocarbon and hydrogen with a first catalyst composition to dealkylate at least part of the C₉₊ aromatic hydrocarbons containing C₂₊ alkyl groups and to saturate the resulting C₂₊ olefins to produce a second feed. The second feed is then contacted with a second catalyst composition under conditions effective to transalkylate at least part of the C₉₊ aromatic hydrocarbons with at least part of the C₆-C₇ aromatic hydrocarbon to produce a first product comprising xylene. Each of the first and second catalyst compositions is substantially free of amorphous alumina.

### PRIORITY

This invention claims priority to and the benefit of U.S. Ser. No. 62/007,556, filed Jun. 4, 2014.

### FIELD OF THE INVENTION

This disclosure relates to transalkylation of heavy (C₉₊) aromatic hydrocarbon feedstocks to produce xylene and either benzene or toluene.

### BACKGROUND OF THE INVENTION

An important source of xylene in an oil refinery is catalytic reformate, which is produced by contacting a mixture of petroleum naphtha and hydrogen with a strong hydrogenation/dehydrogenation catalyst, such as platinum, on a moderately acidic support, such as a halogen-treated alumina. A C₆ to C₈ fraction is separated from the reformate and extracted with a solvent selective for aromatics or aliphatics to produce a mixture of aromatic compounds that is relatively free of aliphatics. This mixture of aromatic compounds contains benzene, toluene and xylenes (BTX), along with ethylbenzene, and can be processed in known manner to recover the xylene.

However, the quantity of xylene available from reforming is limited and so recently refineries have also focused on the production of xylene by transalkylation of heavy (C₉₊) aromatic hydrocarbons with benzene and/or toluene over noble metal-containing zeolite catalysts. For example, U.S. Pat. No. 5,942,651 discloses a process for the transalkylation of heavy aromatics comprising contacting a feed comprising C₉₊ aromatic hydrocarbons and toluene with a first catalyst composition comprising a molecular sieve having a constraint index ranging from 0.5 to 3, such as ZSM-12, and a hydrogenation component under transalkylation reaction conditions to produce a transalkylation reaction product comprising benzene and xylene. The transalkylation reaction product is then contacted with a second catalyst composition which comprises a molecular sieve having a constraint index ranging from 3 to 12, such as ZSM-5, and which may be in a separate bed or a separate reactor from the first catalyst composition, under conditions to remove benzene co-boilers in the product.

One problem associated with heavy aromatics transalkylation processes is catalyst aging since, as the catalyst loses activity with increasing time on stream, higher temperatures tend to be required to maintain constant conversion. When the maximum reactor temperature is reached, the catalyst needs to be replaced or regenerated, normally by oxidation. In particular, it has been found that the aging rate of existing transalkylation catalysts is strongly dependent on the presence in the feed of aromatic compounds having alkyl substituents with two or more carbon atoms, such as ethyl and propyl groups. Thus these compounds tend to undergo reactions such as disproportionation and dealkylation/realkylation to produce C₁₀₊ coke precursors.

To address the problem of C₉₊ feeds containing high levels of ethyl and propyl substituents, US 2009/0112034 discloses a catalyst system adapted for transalkylation of a C₉₊ aromatic feedstock with a C₆-C₇ aromatic feedstock comprising: (a) a first catalyst comprising a first molecular sieve having a Constraint Index in the range of 3-12 and 0.01 to 5 wt. % of at least one source of a first metal element of Groups 6-10; and (b) a second catalyst comprising a second molecular sieve having a Constraint Index less than 3 and 0 to 5 wt. % of at least one source of a second metal element of Groups 6-10, wherein the weight ratio of said first catalyst to said second catalyst is in the range of 5:95 to 75:25. The first catalyst, which is optimized for dealkylation of the ethyl and propyl groups in the feed, is located in front of the second catalyst, which is optimized for transalkylation, when they are brought into contact with a C₉₊ aromatic feedstock and a C₆-C₇ aromatic feedstock in the presence of hydrogen. However, despite these and other advances, there remains a need to further improve the cycle length of the catalysts employed in C₉₊ aromatic transalkylation processes as well as to enhance their capability for processing C₁₀₊ aromatic compounds with good light olefin saturation but low aromatic saturation. Other references of interest include: U.S. Pat. No. 7,485,763; U.S. Pat. No. 5,271,920; U.S. Pat. No. 4,900,529; US 2011/0118520; US 2005/0065017; US 2010/0094068; US 2010/0093520; US 2012/0244049; US 2010/0029467; US 2005/0215838; US 2008/0035525; EP 1586376; EP 1655277; and EP 141514.

### SUMMARY OF THE INVENTION

According to the present disclosure, it is found that the cycle life and aromatic yields in the production of xylene from C₉₊ aromatic hydrocarbons can be improved by reducing or eliminating the alumina conventionally employed as the binder for the molecular sieve catalysts used in existing transalkylation processes. Without wishing to be bound by any theory of operation, it is believed that the alumina binder provides external active sites for coke formation which accelerates catalyst aging and hence reduces cycle time. Thus, in one aspect, the present disclosure relates to a process for producing xylene from C₉₊ aromatic hydrocarbons. The process comprises at least two steps. The first step is contacting a first feed comprising C₉₊ aromatic hydrocarbons, at least one C₆-C₇ aromatic hydrocarbon and hydrogen with a first catalyst composition under conditions effective to dealkylate at least part of the C₉₊ aromatic hydrocarbons containing C₂₊ alkyl groups and to saturate the resulting C₂₊ olefins to produce a second feed. In this first step, the first catalyst composition comprises a first molecular sieve having a Constraint Index of 3 to 12 and a hydrogenation component and the second step is contacting the second feed with a second catalyst composition under conditions effective for transalkylation of at least part of the C₉₊ aromatic hydrocarbons in the second feed with at least part of the C₆-C₇ aromatic hydrocarbon in the second feed to produce a first product comprising xylene. The second catalyst composition comprises a second molecular sieve having a Constraint Index less than 3. In this process, each of the first and second catalyst compositions is substantially free of amorphous alumina. Typically, at least one of the first and second catalyst compositions is substantially binder-free or comprises a silica binder or a binder comprising a crystalline molecular sieve. In addition, the first molecular sieve typically comprises ZSM-5 and the second molecular sieve comprises ZSM-12. The process can further comprise a third step, wherein at least a portion of the first product is contacted with a third catalyst composition under conditions effective to remove benzene coboilers in the first product and produce a second product, wherein the third catalyst composition comprises a third molecular sieve having a Constraint Index of 3 to 12 and is substantially free of amorphous alumina.

In a further aspect, the present disclosure relates to a catalyst system for transalkylating a feed comprising C₉₊ aromatic hydrocarbons to produce xylene. The catalyst system comprises at least three catalyst compositions. The first catalyst composition comprises a first molecular sieve having a Constraint Index of 3 to 12 and a hydrogenation component. The second catalyst composition comprises a second molecular sieve having a Constraint Index less than 3. The second catalyst composition is located downstream of the first catalyst composition when the catalyst system is contacted with the feed. The third catalyst composition comprises a third molecular sieve having a Constraint Index of 3 to 12. The third catalyst composition being located downstream of the second catalyst composition when the catalyst system is contacted with the feed. Each of the first, second and third catalyst compositions is substantially free of amorphous alumina.

### BRIEF DESCRIPTION OF THE DRAWINGS

FIG. 1 is a graph plotting top bed normalized average reactor temperature (NART) and coke make against days on stream (DOS) for the heavy aromatic conversion processes of Example 1 (using a silica-bound PtZSM-5 catalyst) and Comparative Example 1 (using an alumina-bound PtZSM-5 catalyst).

FIG. 2 is a graph of wt % ethyl-aromatic conversion against top bed NART for the heavy aromatic conversion processes of Example 1 and Comparative Example 1.

### DETAILED DESCRIPTION OF THE EMBODIMENTS

As used herein, the numbering scheme for the Periodic Table Groups is as described in Chemical and Engineering News, 63(5), 27 (1985).

The term “aromatic” is used herein in accordance with its art-recognized scope which includes alkyl substituted and unsubstituted mono- and polynuclear compounds.

The term “ethyl-aromatic compounds” means aromatic compounds having an ethyl group attached to the aromatic ring. The term “propyl-aromatic compounds” means aromatic compounds having a propyl group attached to the aromatic ring.

The term “Cₙ” hydrocarbon or aromatic as used herein means a hydrocarbon or aromatic compound having n number of carbon atom(s) per molecule. The term “Cₙ₊” hydrocarbon or aromatic means hydrocarbon or aromatic compound having n or more than n carbon atom(s) per molecule. The term “Cₙ₋” hydrocarbon or aromatic means a hydrocarbon or aromatic compound having less than n carbon atom(s) per molecule.

The term “substantially free” when used in relation to a specific component of a catalyst composition means that the catalyst composition contains less than 1 wt. %, preferably less than 0.15 wt. %, of that component.

“Aromatic ring-loss,” as used herein, is calculated by the following formula: Aromatic Ring loss (%)=(1−total moles of aromatic compounds in product/total moles of aromatic compounds in feed)*100.

The term “benzene co-boiler” means impurities that have a boiling point close to the boiling point of benzene as described at page 393 of Aromatic Hydrocarbons—Advances in Research and Treatment, 2013 Edition, Acton, Q. A. Ed., Scholarly Editions, Atlanta, Ga. (2013).

Described herein is a process for producing xylenes from C₉₊ aromatic hydrocarbons using a series-connected multiple bed catalyst system. Typically, the process employs a first catalyst bed comprising a first catalyst composition selective for the dealkylation of ethyl-aromatic compounds and propyl-aromatic compounds in the C₉₊ aromatic hydrocarbon feed. Downstream of the first catalyst bed is a second catalyst bed comprising a second catalyst composition effective to transalkylate C₉₊ aromatic hydrocarbons with cofed benzene and/or toluene to produce xylenes. In most embodiments, the xylene-containing product of the transalkylation step is then passed to a third catalyst bed which is located downstream of the second catalyst bed and which comprises a third catalyst composition effective to remove benzene co-boilers in the product. In some embodiments, the third catalyst bed is omitted and the process employs the first and second catalysts beds only. Preferably, however, the process employs the first, second, and third, catalyst beds.

Each catalyst bed can be housed in a separate reactor or, where desired, two or more of the catalysts beds can be accommodated in the same reactor. For example, the first, second, and third, catalyst beds can be stacked one on top of the other in a single reactor.

### Feedstock

The aromatic feed used in the present process comprises one or more aromatic hydrocarbons containing at least 9 carbon atoms. Specific C₉₊ aromatic compounds found in a typical feed include mesitylene (1,3,5-trimethylbenzene), durene (1,2,4,5-tetramethylbenzene), hemimellitene (1,2,4-trimethylbenzene), pseudocumene (1,2,4-trimethylbenzene), 1,2-methylethylbenzene, 1,3-methylethylbenzene, 1,4-methylethylbenzene, propyl-substituted benzenes, butyl-substituted benzenes, and dimethylethylbenzenes. Suitable sources of the C₉₊ aromatics are any C₉₊ fraction from any refinery process that is rich in aromatics. This aromatics fraction contains a substantial proportion of C₉₊ aromatics, e.g., at least 80 wt. % C₉₊ aromatics, wherein preferably at least 80 wt. %, and more preferably more than 90 wt. %, of the hydrocarbons will range from C₉ to C₁₂. Typical refinery fractions which may be useful include catalytic reformate, FCC naphtha or TCC naphtha.

The feed to the process also includes benzene and/or toluene. In one practical embodiment, the feed to the transalkylation reactor comprises C₉₊ aromatics hydrocarbons and toluene. The feed may also include recycled/unreacted toluene and C₉₊ aromatic feedstock that is obtained by distillation of the effluent product of the transalkylation reaction itself. Typically, toluene constitutes from 0 to 90 wt. %, such as from 10 to 70 wt. % of the entire feed, whereas the C₉₊ aromatics component constitutes from 10 to 100 wt. %, such as from 30 to 85 wt. % of the entire feed to the process.

The feed to the process will also normally include hydrogen to saturate the C₂₊ olefins generated by the dealkylation reactions occurring in the optional first catalyst bed.

### First Catalyst Bed

When present, the first catalyst bed employed in the present catalyst system contains a first catalyst composition comprising a first molecular sieve having a Constraint Index in the range of about 3 to about 12 and at least one hydrogenation component.

Constraint Index is a convenient measure of the extent to which an aluminosilicate or other molecular sieve provides controlled access to molecules of varying sizes to its internal structure. For example, molecular sieves which provide a highly restricted access to and egress from its internal structure have a high value for the Constraint Index. Molecular sieves of this kind usually have pores of small diameter, e.g., less than 5 Angstroms. On the other hand, molecular sieves which provide relatively free access to their internal pore structure have a low value for the constraint index, and usually pores of large size. The method by which constraint index is determined is described fully in U.S. Pat. No. 4,016,218, which is incorporated herein by reference for the details of the method.

Suitable molecular sieves for use in the first catalyst composition comprise at least one of ZSM-5, ZSM-11, ZSM-22, ZSM-23, ZSM-35, ZSM-48, ZSM-57, and ZSM-58. ZSM-5 is described in detail in U.S. Pat. No. 3,702,886 and Re. 29,948. ZSM-11 is described in detail in U.S. Pat. No. 3,709,979. ZSM-22 is described in U.S. Pat. Nos. 4,556,477 and 5,336,478. ZSM-23 is described in U.S. Pat. No. 4,076,842. ZSM-35 is described in U.S. Pat. No. 4,016,245. ZSM-48 is more particularly described in U.S. Pat. No. 4,234,231 and U.S. Pat. No. 4,375,573. ZSM-57 is described in U.S. Pat. No. 4,873,067. ZSM-58 is described in U.S. Pat. No. 4,698,217.

In one preferred embodiment, the first molecular sieve comprises ZSM-5 and especially ZSM-5 having an average crystal size of less than 0.1 micron, for example such that the ZSM-5 crystals have an external surface area in excess of 100 m²/g as determined by the t-plot method for nitrogen physisorption. Suitable ZSM-5 compositions are disclosed in PCT/US2013/071456, filed Nov. 22, 2013 (which claims priority to U.S. Ser. No. 61/740,908, filed Dec. 21, 2012) and PCT/US2013/071446, filed Nov. 22, 2013 (which claims priority to U.S. Ser. No. 61/740,917, filed Dec. 21, 2012, the entire contents of which are incorporated herein by reference).

Conveniently, the first molecular sieve has an alpha value in the range of about 100 to about 1500, such as about 150 to about 1000, for example, about 150 to about 600. Alpha value is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395.

Generally, the first molecular sieve is an aluminosilicate having a silica to alumina molar ratio of less than 1000. Typically, the silica to alumina molar ratio is from about 10 to about 100.

Typically, the first catalyst composition comprises at least 1 wt. %, preferably at least 10 wt. %, more preferably at least 25 wt. %, and most preferably at least 50 wt. %, of the first molecular sieve. In one embodiment, the first catalyst composition comprises from 55 to 80 wt. % of the first molecular sieve.

In addition to a molecular sieve having a Constraint Index in the range of about 3 to about 12, the first catalyst composition comprises at least one hydrogenation component, such as at least one metal or compound thereof of Groups 6 to 12 of the Periodic Table of the Elements. Suitable hydrogenation components include platinum, palladium, iridium, rhenium, and mixtures and compounds thereof, preferably platinum, rhenium, and compounds thereof. In some embodiments, the first catalyst composition comprises two or more hydrogenation components including a first metal or compound thereof selected from platinum, palladium, iridium, rhenium, and mixtures thereof, and a second metal or compound chosen so as to lower the benzene saturation activity of the first metal. Examples of suitable second metals include at least one of copper, silver, gold, ruthenium, iron, tungsten, molybdenum, cobalt, nickel, tin, and zinc. Conveniently, the first metal is present in the first catalyst in an amount from 0.001 to 1 wt. %, such as from 0.01 to 0.1 wt. %, of the first catalyst and the second metal is present in the first catalyst in amount from 0.001 to 10 wt. %, or 0.1 to 1 wt. %, of the first catalyst.

In some embodiments, the first metal comprises platinum and/or rhenium and the second metal comprises copper and/or tin. In one preferred embodiment, the first metal comprises platinum and the second metal comprises tin, desirably at a molar ratio of platinum to tin from 0.1:1 to 1:1, such as from 0.2:1 to 0.4:1.

The hydrogenation component can be incorporated into the first catalyst composition by any known method, including co-crystallization, ion exchange into the composition to the extent a Group 13 element, e.g., aluminum, is in the molecular sieve structure, impregnated therein, or mixed with the molecular sieve and binder. In some embodiments, ion exchange may be preferred. After incorporation of the hydrogenation component(s), the catalyst composition is usually dried by heating at a temperature of 65° C. to 160° C., typically 110° C. to 143° C., for at least 1 minute and generally not longer than 24 hours, at pressures ranging from 100 to 200 kPa-a. Thereafter, the catalyst composition may be calcined in a stream of dry gas, such as air or nitrogen, at temperatures of from 260° C. to 650° C. for 1 to 20 hours. Calcination is typically conducted at pressures ranging from 100 to 300 kPa-a.

In an embodiment, the hydrogenation component may be treated (such as by sulfiding) to alter the activity of the hydrogenation catalyst.

A method for minimizing the aromatics hydrogenation activity of the catalyst composition is by exposing it to a compound containing an element selected from group 15 or 16 of the Periodic Table of the Elements, preferably N, P, S, O. The group 16 element specifically contemplated is sulfur. A specifically contemplated group 15 element is nitrogen.

Effective treatment is accomplished by contacting the catalyst with a source of sulfur at a temperature ranging from about 200° to 480° C. The source of sulfur can be contacted with the catalyst via a carrier gas, typically, an inert gas such as hydrogen or nitrogen. In a useful embodiment, the source of sulfur is typically hydrogen sulfide.

The catalyst composition can also be treated in situ. For example, a source of sulfur is contacted with the catalyst composition by adding it to the hydrocarbon feedstream in a concentration ranging from about 50 ppmw sulfur to about 10,000 ppmw sulfur. Any sulfur compound that will decompose to form H₂S and a light hydrocarbon at about 490° C. or less will suffice. Typical examples of useful sources of sulfur include carbon disulfide and alkylsulfides such as methylsulfide, dimethylsulfide, dimethyldisulfide, diethylsulfide and dibutyl sulfide. Sulfur treatment can be considered sufficient when sulfur breakthrough occurs; that is, when sulfur appears in the liquid product.

Typically, sulfur treatment is initiated by incorporating a source of sulfur into the feed and continuing sulfur treatment for a few days, typically, up to 10 days, more specifically, from one to five days. The sulfur treatment can be monitored by measuring the concentration of sulfur in the product off gas. During this treatment, the sulfur concentration in the off gas should range from about 20 to about 500 ppmw sulfur, preferably about 30 to 250 ppmw.

Continuously cofeeding a source of sulfur has been found to maintain reduced aromatics hydrogenation activity. The catalyst can be contacted with sulfur during service by cofeeding sulfur to the reactor in varied amounts via the hydrogen stream entering the reactor or the hydrocarbon feedstock. The sulfur can be continuously added to the feedstock throughout the process cycle or the sulfur can be intermittently continuously added in which this sulfur is cofed continuously for a period of time, discontinued, then cofed again.

Such sulfur treatments are effective at any temperature for a metal that is already fully reduced, which can be limited by the thermal decomposition temperature of the sulfiding agent to H₂S.

The first catalyst composition may be self-bound (that is without a separate binder) or may also comprise a binder or matrix material that is resistant to the temperatures and other conditions employed in the present process. Where such a binder or matrix material is present, it is substantially free of amorphous alumina, since it is found that the exclusion of a binder containing amorphous alumina reduces external catalytic sites for coke production and hence increases catalyst cycle length. One preferred binder material for the first catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the first molecular sieve.

Where the first catalyst composition contains a binder or matrix material, the latter may be present in an amount ranging from 5 to 95 wt. % of the total catalyst composition. Typically, the matrix material is present in an amount ranging from 10 to 60 wt. % of the total catalyst composition.

The first catalyst composition may be extruded into particles of any desired shape before being loaded into the first catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to <200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

In embodiments, the first catalyst composition comprises from 10 wt. % to 50 wt % of the total weight of the first, second and third catalyst compositions. For example, the first catalyst composition may comprise from 15 wt % to 35 wt %, of the total weight of the first, second and third catalyst compositions.

In operation, the first catalyst bed is maintained under conditions effective to dealkylate aromatic hydrocarbons containing C₂₊ alkyl groups in the heavy aromatic feedstock and to saturate the resulting C₂₊ olefins. Suitable conditions for operation of the first catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 2 to about 20 hr⁻¹.

### Second Catalyst Bed

The second catalyst bed contains a second catalyst composition comprising a second molecular sieve different from the first molecular sieve. Optionally, the second catalyst bed may contain one or more hydrogenation components.

Desirably, the second molecular sieve has a Constraint Index less than 3 and may comprise at least one of zeolite beta, zeolite Y, Ultrastable Y (USY), Dealuminized Y (Deal Y), mordenite, NU-87, ZSM-3, ZSM-4 (Mazzite), ZSM-12, ZSM-18, MCM-22, PSH-3, SSZ-25, MCM-36, MCM-49, MCM-56, EMM-10, EMM-10-P and ZSM-20. Zeolite ZSM-4 is described in U.S. Pat. No. 3,923,636. Zeolite ZSM-12 is described in U.S. Pat. No. 3,832,449. Zeolite ZSM-20 is described in U.S. Pat. No. 3,972,983. Zeolite Beta is described in U.S. Pat. No. 3,308,069, and Re. 28,341. Low sodium Ultrastable Y molecular sieve (USY) is described in U.S. Pat. No. 3,293,192 and U.S. Pat. No. 3,449,070. Dealuminized Y zeolite (Deal Y) may be prepared by the method found in U.S. Pat. No. 3,442,795. Zeolite UHP-Y is described in U.S. Pat. No. 4,401,556. Rare earth exchanged Y (REY) is described in U.S. Pat. No. 3,524,820. Mordenite is a naturally occurring material but is also available in synthetic forms, such as TEA-mordenite (i.e., synthetic mordenite prepared from a reaction mixture comprising a tetraethylammonium directing agent). TEA-mordenite is disclosed in U.S. Pat. No. 3,766,093 and U.S. Pat. No. 3,894,104. MCM-22 is described in U.S. Pat. No. 4,954,325. PSH-3 is described in U.S. Pat. No. 4,439,409. SSZ-25 is described in U.S. Pat. No. 4,826,667. MCM-36 is described in U.S. Pat. No. 5,250,277. MCM-49 is described in U.S. Pat. No. 5,236,575. MCM-56 is described in U.S. Pat. No. 5,362,697.

Typically, the second catalyst composition comprises at least 1 wt. %, preferably at least 10 wt. %, more preferably at least 25 wt. %, and most preferably at least 50 wt. %, of the second molecular sieve. In one embodiment, the first catalyst composition comprises from 55 to 80 wt. % of the second molecular sieve.

In one preferred example, the second molecular sieve comprises ZSM-12. More preferably, the molecular sieve comprises ZSM-12 having an average crystal size of less than 0.1 micron, such as about 0.05 micron.

In addition to one or more molecular sieves as described above, the second catalyst composition optionally comprises at least one hydrogenation component, such as at least one metal or compound thereof of Groups 6 to 12 of the Periodic Table of the Elements. Suitable hydrogenation components include platinum, palladium, iridium, rhenium and mixtures and compounds thereof, preferably platinum, rhenium and compounds thereof. In some embodiments, the second catalyst composition comprises two or more hydrogenation components including a first metal or compound thereof selected from platinum, palladium, iridium, rhenium and mixtures thereof and a second metal or compound chosen so as to lower the benzene saturation activity of the first metal. Examples of suitable second metals include at least one of copper, silver, gold, ruthenium, iron, tungsten, molybdenum, cobalt, nickel, tin and zinc. Conveniently, the first metal is present in the second catalyst in an amount from 0.001 to 1 wt. %, such as from 0.01 to 0.1 wt. %, of the second catalyst and the second metal is present in the second catalyst in amount from 0.001 to 10 wt. %, 0.1 to 1 wt. %, of the second catalyst.

In some embodiments, the first metal comprises platinum and/or rhenium and the second metal comprises copper and/or tin. In one preferred embodiment, the first metal comprises platinum and the second metal comprises tin, desirably at a molar ratio of platinum to tin from 0.1:1 to 1:1, such as from 0.2:1 to 0.4:1.

The hydrogenation component can be incorporated into the second catalyst composition by any known method, including co-crystallization, ion exchange into the composition to the extent a Group 13 element, e.g., aluminum, is in the molecular sieve structure, impregnated therein, or mixed with the molecular sieve and binder. In some embodiments, ion exchange may be preferred. After incorporation of the hydrogenation component(s), the catalyst composition is usually dried by heating at a temperature of 65° C. to 160° C., typically 110° C. to 143° C., for at least 1 minute and generally not longer than 24 hours, at pressures ranging from 100 to 200 kPa-a. Thereafter, the catalyst composition may be calcined in a stream of dry gas, such as air or nitrogen, at temperatures of from 260° C. to 650° C. for 1 to 20 hours. Calcination is typically conducted at pressures ranging from 100 to 300 kPa-a.

A method for minimizing the aromatics hydrogenation activity of the catalyst composition is by exposing it to a compound containing an element selected from group 15 or 16 of the Periodic Table of the Elements, preferably N, P, S, O. The group 16 element specifically contemplated is sulfur. A specifically contemplated group 15 element is nitrogen.

Effective treatment is accomplished by contacting the catalyst with a source of sulfur at a temperature ranging from about 200° to 480° C. The source of sulfur can be contacted with the catalyst via a carrier gas, typically, an inert gas such as hydrogen or nitrogen. In a useful embodiment, the source of sulfur is typically hydrogen sulfide.

The catalyst composition can also be treated in situ. For example, a source of sulfur is contacted with the catalyst composition by adding it to the hydrocarbon feedstream in a concentration ranging from about 50 ppmw sulfur to about 10,000 ppmw sulfur. Any sulfur compound that will decompose to form H₂S and a light hydrocarbon at about 490° C. or less will suffice. Typical examples of useful sources of sulfur include carbon disulfide and alkylsulfides such as methylsulfide, dimethylsulfide, dimethyldisulfide, diethylsulfide and dibutyl sulfide. Sulfur treatment can be considered sufficient when sulfur breakthrough occurs; that is, when sulfur appears in the liquid product.

Typically, sulfur treatment is initiated by incorporating a source of sulfur into the feed and continuing sulfur treatment for a few days, typically, up to 10 days, more specifically, from one to five days. The sulfur treatment can be monitored by measuring the concentration of sulfur in the product off gas. During this treatment, the sulfur concentration in the off gas should range from about 20 to about 500 ppmw sulfur, preferably about 30 to 250 ppmw.

Continuously cofeeding a source of sulfur has been found to maintain reduced aromatics hydrogenation activity. The catalyst can be contacted with sulfur during service by cofeeding sulfur to the reactor in varied amounts via the hydrogen stream entering the reactor or the hydrocarbon feedstock. The sulfur can be continuously added to the feedstock throughout the process cycle or the sulfur can be intermittently continuously added in which this sulfur is cofed continuously for a period of time, discontinued, then cofed again.

Such sulfur treatments are effective at any temperature for a metal that is already fully reduced, which can be limited by the thermal decomposition temperature of the sulfiding agent to H₂S.

The second catalyst composition may be self-bound (that is without a separate binder) or may also comprise a binder or matrix material that is resistant to the temperatures and other conditions employed in the present process. Where such a binder or matrix material is present, it is preferably substantially free of amorphous alumina, since it is found that the exclusion of a binder containing amorphous alumina reduces external catalytic sites for coke production and hence increases catalyst cycle length. One preferred binder material for the second catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the third molecular sieve. In one preferred embodiment, each of the first and second catalyst compositions includes a silica binder.

The second catalyst composition may be extruded into particles of any desired shape before being loaded into the second catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to <200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

In embodiments, the second catalyst composition comprises from 30 wt % to 90 wt % of the total of the first, second and third catalyst compositions. For example, the second catalyst composition may comprise from 50 wt % to 75 wt % of the total weight of the first, second and third catalyst compositions.

In operation, the second catalyst bed is maintained under conditions effective to transalkylate C₉₊ aromatic hydrocarbons with said at least one C₆-C₇ aromatic hydrocarbon. Suitable conditions for operation of the second catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 1 to about 10 hr⁻¹.

### Third Catalyst Bed

When present, the third catalyst bed employed in the present catalyst system contains a third catalyst composition comprising a third molecular sieve having a Constraint Index in the range of about 3 to about 12. Suitable molecular sieves for use in the third catalyst composition comprise at least one of ZSM-5, ZSM-11, ZSM-22, ZSM-23, ZSM-35, ZSM-48, ZSM-57 and ZSM-58, with ZSM-5 being preferred.

In one preferred embodiment, the third molecular sieve comprises ZSM-5 and especially ZSM-5 having an average crystal size of less than 0.1 micron, for example such that the ZSM-5 crystals have an external surface area in excess of 100 m²/g as determined by the t-plot method for nitrogen physisorption. Suitable ZSM-5 compositions are disclosed in PCT Applications PCT/US2013/071446 and PCT/US2013/071456, both filed on Nov. 22, 2013 the entire contents of which are incorporated herein by reference.

Conveniently, the third molecular sieve has an alpha value in the range of about 100 to about 1500, such as about 150 to about 1000, for example about 150 to about 600.

The third catalyst composition may be self-bound (that is without a separate binder) or may also comprise a binder or matrix material that is resistant to the temperatures and other conditions employed in the present process. Where such a binder or matrix material is present, it is preferably substantially free of amorphous alumina, since it is found that the exclusion of a binder containing amorphous alumina reduces external catalytic sites for coke production and hence increases catalyst cycle length. One preferred binder material for the third catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the third molecular sieve. In one preferred embodiment, each of the first, second and third catalyst compositions includes a silica binder.

Where the third catalyst composition contains a binder or matrix material, the latter may be present in an amount ranging from 5 to 95 wt. % of the total catalyst composition. Typically, in such instances, the matrix is present in an amount ranging from 10 to 60 wt. %, of the total catalyst composition.

The third catalyst composition may be extruded into particles of any desired shape before being loaded into the first catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to <200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

In embodiments, the third catalyst composition comprises up to 25 wt % of the total weight of the first, second and third catalysts compositions. For example, the third catalyst may comprise from 5 wt % to 25 wt %, such as from 5 wt % to 15 wt % of the total weight of the first, second and third catalyst compositions.

In operation, the third catalyst bed is maintained under conditions effective to crack non-aromatic cyclic hydrocarbons in the effluent from the second catalyst bed. Suitable conditions for operation of the third catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 1 to about 50 hr⁻¹.

The invention will now be more particularly described with reference to the following non-limiting Examples and the accompanying drawings.

### Example 1

A silica-bound PtZSM-5 catalyst (50 wt % SiO₂/50 wt % ZSM-5) was prepared from ZSM-5 having an average crystal size of 0.02-0.05 micron. The catalyst contained 0.05 wt % platinum and had an alpha value of 170.

The resultant catalyst was used to convert a heavy aromatics stream comprising 85 wt % of a C₉+ aromatics feed blended with 7 wt % benzene and 8 wt % toluene. Reaction conditions included an initial temperature of 407° C., WHSV of 15, a pressure of 350 psia (2413 kPa) and a hydrogen to hydrocarbon molar ratio of 2.

Before evaluating its performance, the catalyst was de-edged by contacting with the feed at a temperature of 430° C., WHSV of 15, a pressure of 350 psia (2413 kPa) and a hydrogen to hydrocarbon molar ratio of 1 for 3 days.

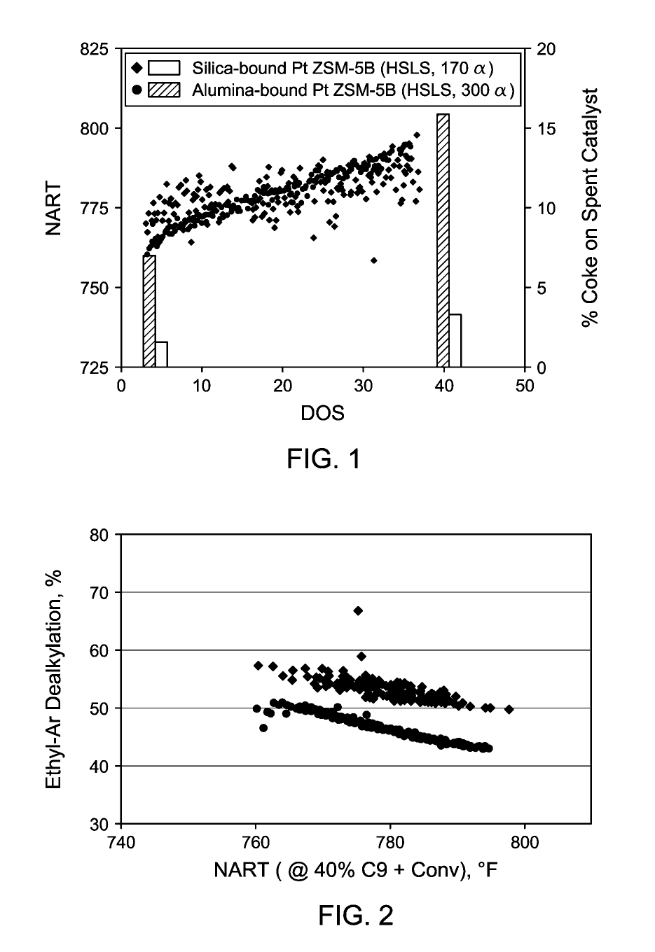

A catalyst aging study was conducted by measuring the normalized average reactor temperature (NART) required to achieve 40% conversion of the C₉ and C₁₀ aromatics in the feed over a period of 40 days. The results are shown in FIG. 1. The ethyl-aromatic conversion activity of the catalyst system was also measured during the 40 day aging study and the results are shown in FIG. 2.

### Comparative Example 1

The process of Example 1 was repeated but with an alumina bound PtZSM-5 catalyst (50 wt % Al₂O₃/50 wt % ZSM-5). Again the ZSM-5 had an average crystal size of 0.02-0.05 micron and the catalyst contained 0.05 wt % platinum. The alpha value of the catalyst was 300. The results of the aging study are again shown in FIGS. 1 and 2 from which it will be seen that the silica bound catalyst exhibited reduced aging, lower coke make and higher dealkylation activity than the alumina bound catalyst system.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

All documents described herein are incorporated by reference herein for purposes of all jurisdictions where such practice is allowed, including any priority documents and/or testing procedures to the extent they are not inconsistent with this text, provided however that any priority document not named in the initially filed application or filing documents is NOT incorporated by reference herein. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including”. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

What is claimed is:

1. A process for producing xylene from C₉₊ aromatic hydrocarbons, the process comprising:
(a) contacting a first feed comprising C₉₊ aromatic hydrocarbons, at least one C₆-C₇ aromatic hydrocarbon and hydrogen with a first catalyst composition under conditions effective to dealkylate at least part of the C₉₊ aromatic hydrocarbons containing C₂₊ alkyl groups and to saturate the resulting C₂₊ olefins to produce a second feed, wherein the first catalyst composition comprises a first molecular sieve having a Constraint Index of 3 to 12 and a hydrogenation component; and
(b) contacting the second feed with a second catalyst composition under conditions effective for transalkylation of at least part of the C₉₊ aromatic hydrocarbons in the second feed with at least part of the C₆-C₇ aromatic hydrocarbon in the second feed to produce a first product comprising xylene, wherein the second catalyst composition comprises a second molecular sieve having a Constraint Index less than 3;
wherein each of the first and second catalyst compositions is substantially free of amorphous alumina.

2. The process of claim 1, wherein at least one of the first and second catalyst compositions is substantially binder-free.

3. The process of claim 1, wherein at least one of the first and second catalyst compositions comprises a silica binder or a binder comprising a crystalline molecular sieve.

4. The process of claim 1, wherein at least one of the first and second catalyst compositions comprises a silica binder.

5. The process of claim 1, wherein the first molecular sieve comprises ZSM-5.

6. The process of claim 1, wherein the first molecular sieve comprises ZSM-5 crystals having an external surface area in excess of 100 m²/g as determined by the t-plot method for nitrogen physisorption.

7. The process of claim 1, wherein the second molecular sieve comprises ZSM-12.

8. The process of claim 1, wherein the second catalyst composition is substantially free of amorphous alumina.

9. The process of claim 1, wherein the second catalyst composition also comprises a hydrogenation component comprising at least one metal or compound thereof of Groups 6 to 12 of the Periodic Table of the Elements.

10. The process of claim 1, further comprising:
(c) contacting at least part of the first product with a third catalyst composition under conditions effective to remove benzene coboilers in the first product and produce a second product, wherein the third catalyst composition comprises a third molecular sieve having a Constraint Index of 3 to 12; and
(d) recovering xylene from the second product.

11. The process of claim 10, wherein the third catalyst composition is substantially free of amorphous alumina.

12. The process of claim 10, wherein the third catalyst composition is substantially binder-free or comprises a silica binder or a binder comprising a crystalline molecular sieve.

13. The process of claim 10, wherein the third catalyst composition comprises a silica binder.

14. The process of claim 10, wherein the third molecular sieve comprises ZSM-5.

15. The process of claim 10, wherein the third molecular sieve comprises ZSM-5 crystals having an external surface area in excess of 100 m²/g as determined by the t-plot method for nitrogen physisorption.

16. The process of claim 10, wherein each of the first, second and third catalyst compositions comprises a silica binder.

17. A catalyst system for transalkylating a feed comprising C₉₊ aromatic hydrocarbons to produce xylene, the catalyst system comprising:
(i) a first catalyst composition comprising a first molecular sieve having a Constraint Index of 3 to 12 and a hydrogenation component;
(ii) a second catalyst composition comprising a second molecular sieve having a Constraint Index less than 3, the second catalyst composition being located downstream of the first catalyst composition when the catalyst system is contacted with the feed; and
(iii) a third catalyst composition comprising a third molecular sieve having a Constraint Index of 3 to 12, the third catalyst composition being located downstream of the second catalyst composition when the catalyst system is contacted with the feed,
wherein each of the first, second and third catalyst compositions is substantially free of amorphous alumina.

18. The catalyst system of claim 17 and comprising from 15 wt % to 35 wt % of the first catalyst composition, from 50 wt % to 75 wt % of the second catalyst composition, and from 5 wt % to 25 wt % of the third catalyst composition, based on the total weight of the first, second and third catalyst compositions.

19. The process of claim 1, wherein the first molecular sieve comprises ZSM-5 and the second molecular sieve comprises ZSM-12.

20. The process of claim 19, wherein the second catalyst is ZSM-12.

21. The process of claim 19, wherein the third molecular sieve comprises ZSM-5.

22. The catalyst system of claim 17, wherein the first catalyst is ZSM-5, the second catalyst is ZSM-22 and the third catalyst is ZSM-5.
